(19) United States
(12) Patent Application Publication
(10) Pub. No.: US 2022/0304656 A1
(43) Pub. Date: Sep. 29, 2022

(54) DEVICE FOR AND METHOD FOR PREPARING OF A TREATMENT OF A PATIENT WITH HIGH-INTENSITY FOCUSED ULTRASOUND

(71) Applicant: THERACLION SA, Malakoff (FR)

(72) Inventors: Jérémie ANQUEZ, Paris (FR); Anthony GRISEY, Saint Cyr l'Ecole (FR)

(73) Assignee: THERACLION SA, Malakoff (FR)

(21) Appl. No.: 17/619,678

(22) PCT Filed: Jun. 17, 2020
(86) PCT No.: PCT/EP2020/066775

§ 371 Date: Dec. 16, 2021

(30) Foreign Application Priority Data
Jun. 19, 2019 (IB) PCT/IB2019/000708

(51) Int. Cl. A61B 8/00; A61B 34/20

(52) U.S. Cl. CPC A61B 8/4245; A61B 34/20; A61B 2090/378

(57) ABSTRACT

A device (1) and method to treat a patient (P) with High-Intensity Focused Ultrasound (HIFU), wherein the device (1) performs a movement of an imaging device (4) along a longitudinal axis (8a, 8b) while acquiring images.

The invention relates to a device for treating a patient with High-Intensity Focused Ultrasound (HIFU) and a method to prepare such a treatment, in particular according to the independent patent claims.

HIFU treatments enable the non-invasive ablation of anatomical targets in the body. They are usually guided based on an imaging modality such as ultrasonography, in particular B-mode imaging.

In several devices, an ultrasonography imaging transducer is embedded in a treatment head which also comprises the therapy transducer, as e.g. described in WO 2006/129045.

Depending on the clinical indication and the treatment protocol, the targeted tissue can be difficult to visualize, which can make targeting complex. For example, if tumescent anaesthesia is used in the context of a treatment of a varicose vein guided by ultrasound (US), the fluid compresses the vein, which makes it difficult to distinguish it from the surrounding tissue. In particular, the target may be nearly invisible in some 2D planes.

It is thus an object of the present invention to overcome the disadvantages of the prior art, in particular to provide a device and a method which facilitate the targeting of structure to be treated. In particular the device and method are useful for targeting collapsed structures by ultrasound (US)

This and other objects are solved by a device and a method according to the independent claims of the invention.

The device for treatment of a patient by HIFU according to the invention comprises a treatment head that includes a unit for emission of HIFU pulses, and an imaging device having a probe, preferably an imaging device able to perform B-mode imaging. The imaging probe of the imaging device is preferably arranged in the treatment head. The device further comprises a control unit for controlling the movement of the probe. The control unit is adapted to carry out a movement of the probe with respect to a target during the operation of the imaging device.

It is possible to move the probe by moving the treatment head but alternatively also by moving the probe individually with respect to the head.

The invention allows a way of estimating the position of a target based on information acquired where it is visible.

When looking for an object which is barely visible in B-mode imaging with a hand-probe, the natural gesture of the practitioner consists in moving the probe forward and backward orthogonally to the imaging plane.

In particular, this applies to the case of a collapsed vein in transverse view: the practitioner moves the probe back and forth along the longitudinal axis of the vein. This movement enables to (1) differentiate the tubular structure from the roughly round local heterogeneities and (2) mentally follow the path of the tubular structure from the planes where it is visible to the planes where it is less or not visible. The present invention allows to improve targeting in the treatment with HIFU by proposing a robotic movement of the probe similar to such natural gesture used in B mode imaging.

In a preferred embodiment, the control unit is adapted to allow a user-controlled movement and/or to carry out a movement of the probe which approximately follows one of the following axes:
Axis orthogonal to the current imaging plane
Axis parallel to the main axis of the target
Projection of the main target axis in a plane orthogonal to the main ultrasound propagation axis
Projection of the main target axis in a plane parallel to the skin surface.

In general, the device may be adapted such that only one user action is required to perform the movement as described above. For example, one button (or any other trigger described herein) may be used to trigger the movement. The movement may then be performed automatically and may be any of the movements described herein. Particularly preferably, the treatment head stops for 0.5 seconds before moving back to the initial position.

Alternatively or additionally, the device may allow and/or require several user actions. For example, the device may require a first trigger to move the treatment head away from a first position, and a second trigger to move back to the initial position. The movement may also comprise pauses, either automatic stops by the device or pauses triggered by the user.

The device may be adapted such that the control does not allow any treatment pulses to be delivered during movement of the treatment head. At least one image may be acquired during the movement.

Typically, the movement has duration of less than 20 seconds, preferably less than 10 seconds, particularly preferably less than 5 seconds.

Particularly preferably, the control is adapted to maintain acoustic coupling, for example by keeping a constant force applied to the tissue by the treatment head, while performing a movement. This is particularly advantageous if the movement follows a trajectory along the longitudinal axis of the target.

To this end, the device, preferably the treatment head, may comprise a force sensor.

Additionally or alternatively, acoustic coupling is maintained by using a balloon filled with fluid and connected to a fluid circulation system enabling to modify the volume of fluid inside the balloon (as known in the art). Preferably, the fluid pressure in the balloon is monitored. The pressure may be kept substantially constant during the movement in order to automatically adapt to the anatomy.

Preferably, the pressure is not kept constant during the movement, but it is decreased during the movement away from the target while maintaining acoustic coupling, to allow for the target visibility to be enhanced when arriving at the farthest position.

For example, the probe may be embedded into the treatment head and the part of the device holding the treatment head can be switched into a mode, where the current position (including translation and rotation) is memorized as a base position. Manual displacements of the treatment head, optionally restricted to some degrees of freedom, can be performed away from this base position, but when the treatment head is released, at least one component of the position automatically comes back to the same value as in the base position. For example, this can be achieved using a spring-like effect on all coordinates. Preferably, the device forbids triggering a pulse while the treatment head is not at its base position.

Preferably, the control unit limits the movement to a displacement along one of the aforementioned axes.

In particular, the control unit may be adapted such that at least one type of movement which approximately follows one of the aforementioned axes can be triggered by a HIFU user.

In a preferred embodiment, the control unit is adapted to store at least one reference position in a memory. The control unit is then further adapted to trigger a movement of the probe to the reference position.

In another preferred embodiment, the control unit is adapted to only allow a movement, in particular a user-controlled movement, of the probe when no pulse, for example a HIFU treatment pulse, is being emitted. In particular, the control unit may allow a movement of the probe in between pulses.

Preferably, the treatment head may move along a predefined trajectory during the pulses to appropriately spread the energy. Alternatively or additionally, an automatic algorithm may be responsible for adapting this trajectory based on real-time temperature feedback or for tracking the target based on automatic image processing. In particular, such automated movement may still be performed, even during emission of a pulse, if the device is adapted to only allow user-controlled movement when no pulse is being emitted.

In a preferred embodiment, the control unit is adapted to carry out a movement of the probe away from an initial position at a first speed followed by a return to the initial position preferably at a second, slower sped.

The movement may be triggered automatically or manually by a user. In a preferred embodiment, the device comprises a user interface to trigger a movement of the probe away from the current position.

In a particularly preferred embodiment, the user interface comprises at least one actuator, e.g. a monostable button, to trigger a movement of the probe away from the current position wherein the control is preferably adapted to trigger a movement of the probe back to its initial position or the saved reference position when the user releases the button.

Preferably, the movement of the probe, in particular the movement back to the initial position of the probe after an initial movement, happens at a pace such that the user can visually follow the target on the image (e.g. with a speed <20 mm/s).

Alternatively, a button may be used wherein manual pressing of the button triggers the movement to the reference position. While these aspects are explained with reference to a button, it will be understood that actuation can be made with any kind of actuators, e.g. also software-based actuators.

Alternatively, it is also possible to have a control such that when the user releases the button, the probe stays where it is. Preferably the user can then place a marker on the screen of the imaging device to indicate the position of the vein or of any other structure. Then the return movement can be triggered by pressing the same or another button or actuator.

More precisely, at least one actuator can be used to trigger a movement of the probe away from the current position, in particular to a reference position, approximately along the one of the aforementioned axes. When the user releases the button, the probe goes back to its initial position, in particular to the reference position.

In general, a reference position may refer to a position that is to be treated, i.e. where at least one HIFU pulse is planned to be delivered.

Alternatively, the movement back to the reference position may be performed automatically.

Additionally or alternatively, the reference position may also be corrected, i.e. the movement may be away from the reference position and back to a corrected reference position.

A corrected reference position may, in particular, be advantageous if the movement away from the reference position reveals that the targeted tissue, for example a vein, is displaced from the reference location. In this case, the reference position may be corrected by the determined displacement in order to treat the targeted tissue.

Thus, a corrected reference position may preferably refer to a position in the same imaging plane as the reference position, but with a lateral and/or vertical displacement of the focal point within said plane and/or a plane perpendicular to the direction of the movement.

Preferably, the movement away from the reference position is performed to a position that has not been completely treated, in particular not treated at all.

In a further preferred embodiment, the user interface comprises two buttons to trigger a movement of the probe, preferably one for each direction along a chosen axis. These buttons can be either physical, for example a footswitch, or virtual, for example a touchscreen. Preferably, at least one button is selected from the group of a physical or virtual button.

In a preferred embodiment, the user interface comprises a drag-and-drop control on a screen. In particular, drag-and-drop shall include a mechanism where while a trigger is actuated, for example a virtual button or item clicked, the movement of the head is controlled by the user. When the trigger is released, movement stops.

In a preferred embodiment, the control unit is adapted to carry out an oscillatory movement of the probe approximately along one of the aforementioned axes.

Preferably, the control unit is adapted to carry out
one or predefined number of oscillations around an initial position. It may also be adapted to carry out a finite number of damped oscillations around a current position. The damped oscillation may in particular include any oscillation with a decreasing amplitude over time. It is also possible to carry out continuous oscillations around an initial position until a predetermined criterion is fulfilled, in particular until a button is released.

In particular, a physical or virtual button is used to trigger an oscillatory movement of the probe roughly along one of the aforementioned axes. Possible oscillatory movements include, but are not limited to:
one or a predefined number of oscillations around the current position, for example with a sinusoidal speed
a finite number of damped oscillations around the current position, for example a damped sinusoidal speed
continuous oscillations around the current position until the button is released.

In a preferred embodiment, the control unit is adapted to carry out an oscillatory movement with an amplitude greater than 1 mm, preferably greater than 1 cm. The amplitude may be fixed or adjustable by the user.

In another preferred embodiment, the control unit is adapted to carry out a movement along an at least partially curved trajectory.

In a particularly preferred embodiment, the device is further designed so the movement is not performed along a straight line but along a trajectory which is either defined by the user (for example drawn on a touchscreen) or automatically computed based on the knowledge of the anatomy. For example, if the position of the targeted vein is known in several orthogonal planes, an interpolation of these positions can define a trajectory along which the probe is moved.

In a preferred embodiment, the device comprises at least one of an input interface for definition of the trajectory by the user and a calculation unit for automatically computing the trajectory, preferably based on the knowledge of the anatomy.

In a preferred embodiment, the control unit is adapted to carry out a movement with an average displacement speed comprised between 0.1 mm/s and 100 mm/s, preferably between 0.5 and 30 mm/s.

In particular, the speed of the movement is not necessarily constant over time and/or along the trajectory. Preferably, the speed is adapted to be compatible with the acquisition time of the images so that the spatial interval between two images is small compared to desired precision. For example, if the embedded hardware requires 50 ms to acquire an image and the spatial step between two images shall be smaller than 0.25 mm, the displacement speed shall be smaller than 5 mm/s.

In an alternative embodiment, the control unit is adapted to carry out the movement by a control representing the coordinates of the probe, in particular along an axis substantially orthogonal to the imaging plane. For example, the control may comprise an electronic and/or mechanical control such as a joystick, or can be purely virtual on a screen or user interface. Alternatively or additionally, the control may comprise a representation of an accessible range along the axis and is adapted such that the user can click on a position to move the treatment head to this position. Preferably, at least one reference position is defined for which, when the user clicks in the vicinity of this reference position, the control unit moves the treatment head to said reference position. A reference position may, in particular, be a site where HIFU pulses are planned to be delivered. For example, a discrete set of reference positions may be located on the axis orthogonal to the imaging plane. When the user clicks on a position on this axis, the software computes the closest reference position from the discrete set of positions and moves the treatment head to this position. This enables to move the treatment head away from the current position to see the vein and to come back to one of the reference positions to deliver the pulses with a controlled spacing. The probe and/or the treatment head may perform an action selected from the group of treatment step, HIFU emission and imaging step after the movement away from the vein and/or before the movement back to one of the reference positions.

In another preferred embodiment, the movement is commanded by a control representing the coordinates of the probe, which enables the user to control the movement as if he was holding a hand-probe, for example by a drag-and-drop gesture. Preferably, the control limits the movement to a displacement along one of the aforementioned axes.

Preferably, in embodiments where the movement back to the position where the next pulse will be delivered is not triggered automatically, the user can perform some actions before triggering the return to the position where the next pulse will be delivered. Such actions include, but are not limited to, changing some characteristics of the live image (e.g. switching from B-mode to duplex imaging) and/or moving the probe and/or the treatment head. For example, this can be done in a case where the imaging probe is embedded into the treatment head and the user can control the position of the probe along an axis substantially corresponding to the longitudinal axis of the vein. Once in a position where the user can see the vein, the user can move the treatment head to place the HIFU focus on the target (e.g. using dedicated controls). Then, he can rotate the treatment head along the main ultrasound propagation axis to refine a longitudinal view of the target, which serves to define the new displacement axis as the axis of the longitudinal plane which is orthogonal to the main ultrasound propagation axis. Then, a movement is triggered along this new axis to a position where a sonication should be delivered. In this position, the focus is approximately on the target, and little or no position adjustments are necessary.

In a preferred embodiment, the device comprises a range limiter for limiting the movement of the probe, in particular for limiting the spatial range and/or the range of coordinates the probe can access.

In a particularly preferred embodiment, the range limiter is defined by mechanical limits of a holding arrangement for holding the probe or definable through a user interface, preferably by moving a holding arrangement for holding the probe to the extreme points of image acquisitions.

The treatment head which comprises the imaging probe can be provided with a balloon which defines a cavity for receiving a coupling fluid. Most preferably a pressure feedback loop may ensure proper adaptation to the anatomy. In particular, this shall maintain acoustic coupling throughout the movement.

In a preferred embodiment, the probe is moved in real time using a control.

In an alternative preferred embodiment which will be described in more detail below, the probe is not moved when viewing, but a movement of the probe is simulated virtually by navigating with the control within a set of images pre-acquired with a moving probe.

The device can be adapted to associate the collected images to a coordinate along the trajectory and comprises a display adapted to display the image corresponding to a given coordinate.

In a preferred embodiment, the control unit is further adapted to synchronize the probe movement and image acquisition with positions of the slices where the HIFU pulses will be delivered, so that images are acquired at these positions.

Thus, if the position of the slices where the HIFU pulses will be delivered is known before the acquisition, the probe movement and image acquisition are preferably synchronized so that images are acquired at these positions.

Thus, the collected images are preferably associated to a coordinate along the trajectory and the interface allows for displaying the image corresponding to a given coordinate. Preferably, it is possible to trigger an acquisition at any time during the treatment.

The device can further comprise a navigation control for navigating within an acquired set of images, preferably a physical or a virtual navigation control. Most preferably, the user interface enables to place at least one marker on the images of the set of acquired images. Even more preferably, the marker is also displayed on the live and/or frozen image. The virtual navigation control may, for example be a slider on the user interface or any of the other controls previously described in the context of actual movements of the probe.

In particular, the marker may be appropriately shifted if the two images are not acquired in the same exact position. For example, after the acquisition, the user may have moved the treatment head or the probe to place the focus deeper to target the expected location of the vein. In this case, the live image is acquired in a frame of reference which is shifted compared to the set of acquired images. Thus, when displaying the acquired images, they are preferably shifted to be consistent with the live image.

In an alternative embodiment, any robotic movement performed induces a corresponding shift on the displayed set of acquired images so the centers of both views correspond to the same position in the anatomy. This may require to crop the images displayed in the set of acquired images.

In another particularly preferred embodiment, the display comprises a first dedicated area for display of the acquired images and navigation and a second dedicated area for display of an image of the zone where a sonication has to be delivered. Preferably, the slice displayed in the live view can be marked on the control. Display of the acquired images and navigation is done in a dedicated part of the interface, while the live-image or a frozen image of the zone where a sonication has to be delivered is displayed in a “live-view”.

In yet another particularly preferred embodiment, the display comprises a shared area for displaying the acquired images and for display of an image of the zone where a sonication has to be delivered. When the virtual position of the probe is set at the actual position of the probe, the live image of the zone where a sonication has to be delivered is displayed. However, when the virtual position of the probe is set at another position, the corresponding image from the set of acquired images is displayed.

In a preferred embodiment, the navigation control is monostable such that, when it is released, the virtual position of the probe goes back to the actual position of the probe. The movement back to the actual position of the probe is preferably performed at a low speed that can be visually followed by a user, in particular corresponding to lower than 20 mm/s in real space.

In an alternative embodiment, the control unit is adapted to carry out the movement by a control representing the coordinates of the probe, in particular along an axis substantially orthogonal to the imaging plane. For example, the control may comprise an electronic and/or mechanical control such as a joystick, or can be purely virtual on a screen or user interface. Alternatively or additionally, the control may comprise a representation of an accessible range along the axis and is adapted such that the user can click on a position to move the treatment head to this position. Preferably, at least one reference position is defined for which, when the user clicks in the vicinity of this reference position, the control unit moves the treatment head to said reference position. A reference position may, in particular, be a site where HIFU pulses are planned to be delivered. For example, a discrete set of reference positions may be located on the axis orthogonal to the imaging plane. When the user clicks on a position on this axis, the software computes the closest reference position from the discrete set of positions and moves the treatment head to this position. This enables to move the treatment head away from the current position to see the vein and to come back to one of the reference positions to deliver the pulses with a controlled spacing. The probe and/or the treatment head may perform an action selected from the group of treatment step, HIFU emission and imaging step after the movement away from the vein and/or before the movement back to one of the reference positions.

Preferably, the control unit is adapted to save at least two positions of the treatment head. In particular, the position shall include the Cartesian coordinates and the orientation of the treatment head.

In the present disclosure, the features are described to ease targeting. A person skilled in the art will, however, recognize that they can also be used to estimate the position of some sensitive structures which may be difficult to see (for example, nerves).

The invention is further directed at a method of preparing a treatment of a patient with HIFU. Preferably, the method is performed using a device a disclosed herein. A treatment shall be understood as the emission of at least one HIFU pulse.

The method of preparing a treatment of a patient with HIFU according to the invention comprises the steps performing a movement with a treatment head away from a target site. At the site away from the target site, at least one image is acquired. Based on the at least one acquired image, a target in the vicinity of the target site is defined. The treatment is then moved back to the target site. Optionally, a HIFU pulse can be emitted at the target site.

A target site shall be understood as a position of the treatment head intended for treatment. In particular, it shall comprise the position in the room and the orientation of the treatment head.

For example, the treatment head may be positioned at a target site where a vein is to be treated. Due to low visibility, however, the user cannot recognize the vein on an ultrasonic image. Thus, the treatment head is moved away to a different position where the vein is visible. Based on the position and anatomy of the vein on the image, the user can extrapolate the position of the vein relative to the target site. Thus, upon moving the treatment head back, a HIFU pulse can be emitted with higher precision.

In particular, a marker can be used to mark the target based on the acquired image.

Preferably, the movement away from the target site comprises at least one of a translational movement along the main ultrasound propagation axis, a translational movement along an axis perpendicular to the main ultrasound propagation axis, and a rotational movement around an axis through the focal point.

A rotational movement shall be understood as a movement of the treatment head wherein the focal spot remains at the same place in the patient's anatomy. In particular, the treatment head is rotated around an axis that intersects with the focal point such that the treatment head points at the focal spot throughout the movement.

Preferably, a rotational movement is carried out about an axis parallel to the longitudinal axis of the target. Additionally or alternatively, a translational movement along an axis parallel to the longitudinal axis of the target is performed.

Preferably at least one of the movements of the treatment head is performed automatically. For example, the movement away from the target site can be performed by a user manually, and a device automatically saves the initial position and moves back to it. Additionally or alternatively, the movement away from the target site may also be performed automatically.

An alternative method comprises the step of acquiring a collection of two-dimensional images prior to or during treatment. i.e. between emission of pulses. The image acquisition comprises the steps of positioning a treatment head on the patient, defining a longitudinal direction, wherein this direction preferably corresponds to the main axis of the target or to a projection of this main axis on a plane which is orthogonal to the main ultrasound propagation axis or parallel to the skin, and performing an automatic controlled movement along the longitudinal axis to acquire a set of images which are preferably orthogonal to the longitudinal axis.

In a preferred embodiment, the treatment head which comprises the imaging probe is used to acquire a collection of 2D images prior to or during treatment, i.e. between emission of treatment pulses.

In a preferred embodiment, the movement of the probe comprises a rotation along an axis which is preferably orthogonal to the main ultrasound propagation axis. This rotation may or may not be combined with a translation of the probe.

In a preferred embodiment, the probe is arranged within the treatment head and the control unit is adapted to hold the treatment head to maintain the focus of the HIFU transducer at the same position while enabling rotations of the treatment head.

In a further preferred embodiment, when the user releases the treatment head, it does not come back to its initial rotational or axial position and it memorizes the new rotation coordinates of the treatment head as a visualization angle.

In particular, an axial or rotational position shall be understood as a value for at least one coordinate. For example, a position may fully characterize by it coordinates along 3 axes (x, y, z) and the rotational angles along 3 axes (psi, theta, phi). It is of course possible to save a reference position containing a complete set of coordinates, such as (x0, y0, z0, psi0, theta0, phi0). Thus, the probe may be displaced from the current position (x1, y1, z1, psi1, theta1, phi1) to the position (x0, y0, z0, psi0, theta0, phi0). If a movement had been triggered from another position (x2, y2, z2, psi2, theta2, phi2), the treatment head would also have gone to (x0, y0, z0, psi0, theta0, phi0) as well.

However, it is also possible and shall be encompassed in the meaning of reference position herein that only a subset of coordinates is saved. For example, a reference position may only comprise an angle such as (psi0). If the treatment head is positioned at (x1, y1, z1, psi1, theta1, phi1) and a movement back to the reference position is triggered, the movement results in (x1, y1, z1, psi0, theta1, phi1). If the movement from another position (x2, y2, z2, psi2, theta2, phi2), it would have gone to (x2, y2, z2, psi0, theta2, phi2).

Alternatively, during rotation the focus is not maintained at the same position but along an axis or within a plane, to also allow for translating the treatment head. In a preferred embodiment, this is combined with a spring-like effect as described above. For example, the user can set the device in a mode where i) free rotation is allowed around an axis through the focus and ii) the treatment head can be moved along an axis substantially parallel to the vein longitudinal axis with a spring-like effect. When the user releases the treatment head, the rotational orientation does not change but the treatment comes back to its initial position along the allowed displacement axis.

In a further preferred embodiment, the focus can be automatically or manually translated in the current imaging plane, where the target is visible, to appropriately place the focus to deliver the pulse onto the target. Additionally, a pulse may be delivered. For example, if targeting is made based on B-mode imaging, when the probe is not orthogonal to the vein wall, the ultrasound beam generated by the imaging probe and reflected by the vein wall is reflected away from the probe. Thus, only little signal is detected, and the vein wall is barely visible. Conversely, if the probe is orthogonal to the vein wall, the imaging beam is reflected onto the imaging probe and the signal is good, thus, the vein is visible. Thus, for example, if the user barely sees the vein, the user can set the device to allow a rotational movement the treatment head to find the proper visualization angle. Then, since this angle corresponds to a situation where the skin is substantially orthogonal to the main HIFU propagation axis, it corresponds to a nearly optimal position to deliver a pulse, since energy transmission is high.

Alternatively, another set of rotation coordinates is memorized to define a treatment angle, where at least one pulse is delivered. For example, the user approximately places the treatment head so the focus is approximately on the target. Then, he sets the device to allow for rotating the treatment head and rotates the treatment head to an angle where he can easily see the vein. This position is memorized as the visualization angle. For example, if duplex is used for targeting, this may correspond to a situation where the main propagation axis of the imaging beam is not orthogonal to the vein, to have some Doppler information. Then, the user moves the focus to appropriately target the vein. Then the user sets the device to allow for rotating the treatment head again and rotates it to be in the treatment angle, for example to have the main HIFU propagation axis orthogonal to the skin. The corresponding rotation coordinates are memorized and the user delivers a pulse.

In a preferred embodiment, the user can trigger an automatic movement to place the treatment head in the visualization angle or in the treatment angle when needed. He can also redefine them if needed.

Note that, the center of rotation is not necessarily an axis around the focal spot but that it may be around another axis. For example, if the focus must be placed 1 mm deeper than the vein, the center of rotation can be 1 mm above the position of the focus.

In the following, the invention is described in detail with reference to the following figures, which show:

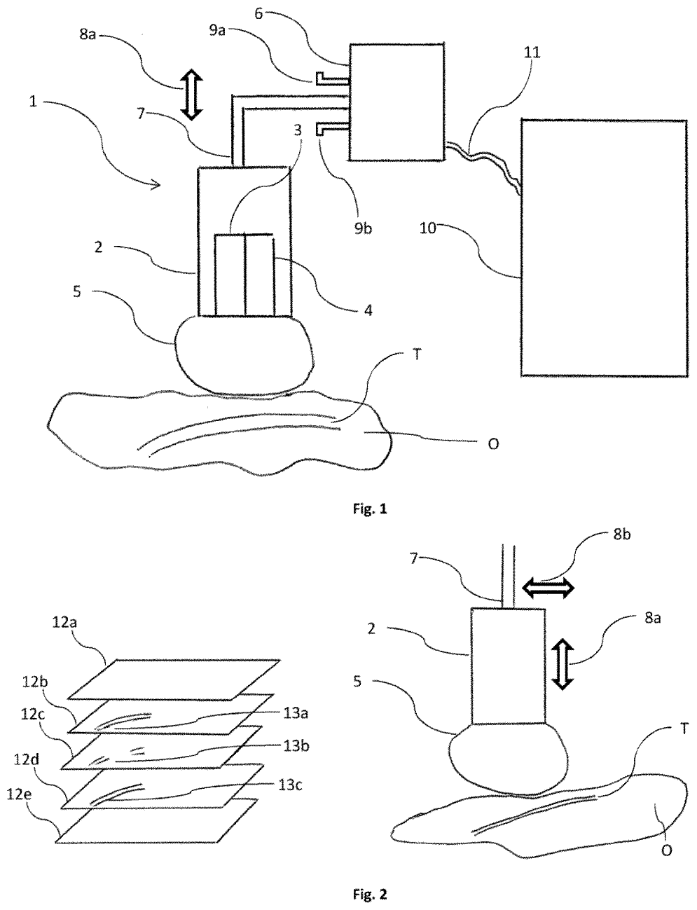

FIG. 1: a schematic representation of the device according to the present invention

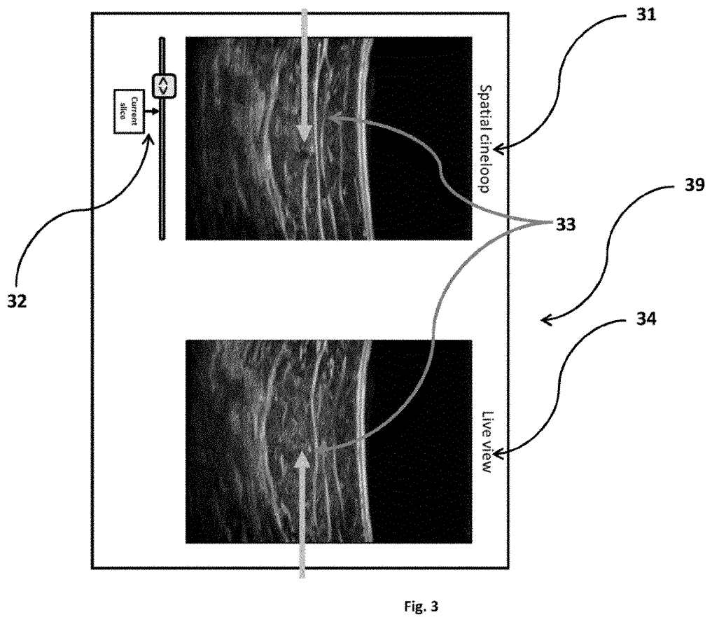

FIG. 2: a schematic representation of the method carried out with the present invention

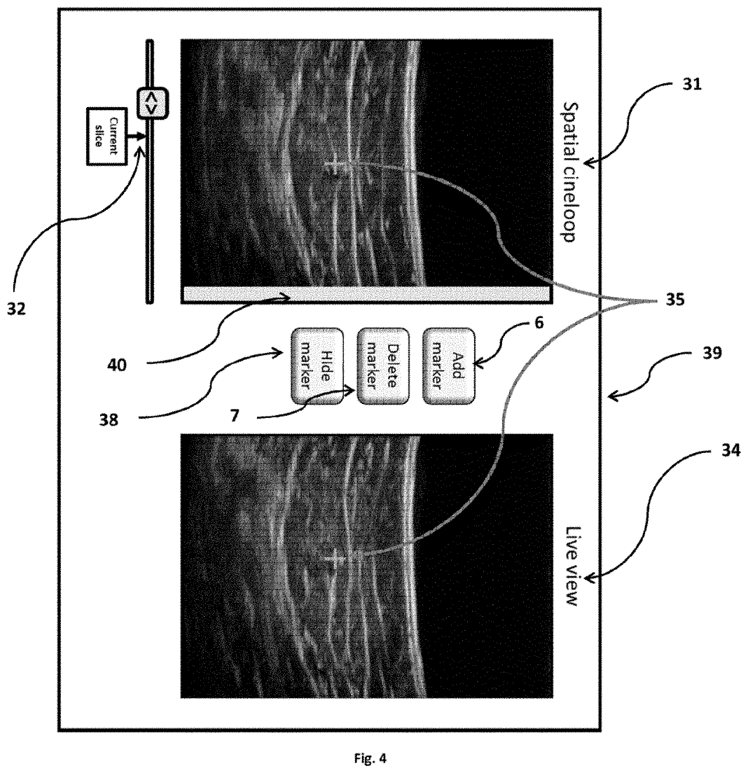

FIG. 3: an example of a display with a set of acquired images and a live view.

FIG. 4: an example of a user interface.

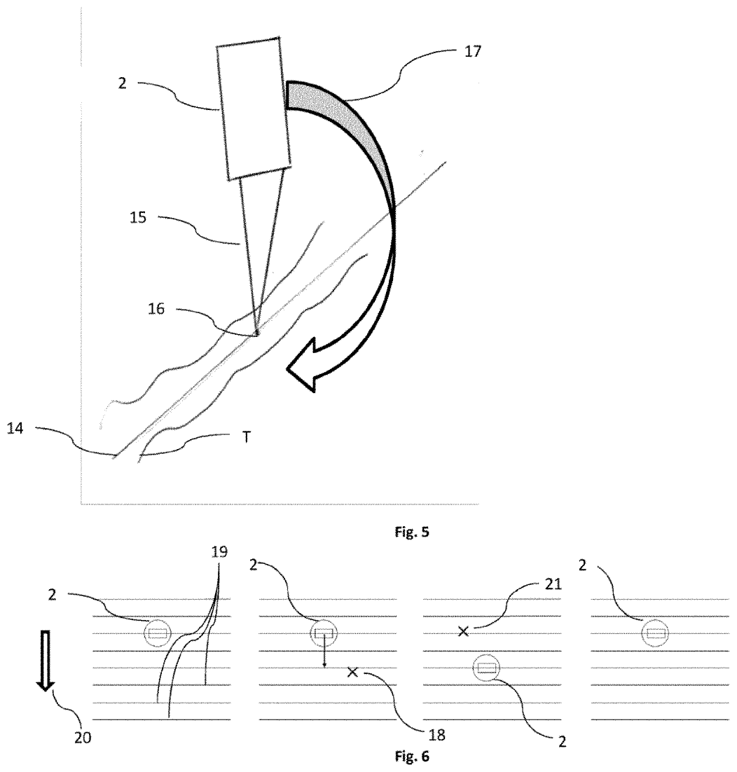

FIG. 5: a schematic illustration of a rotational movement.

FIG. 6: a schematic illustration of a user interface with saved reference positions.

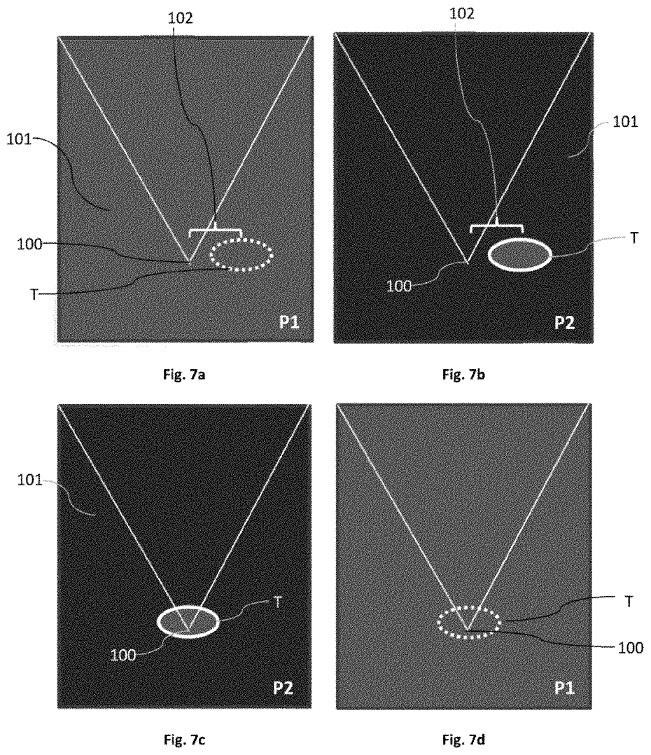

FIGS. 7a-7d: a schematic movement away from a reference position and back to a corrected reference position.

FIG. 1 shows schematically a device 1 according to the invention to treat a patient with HIFU. The device 1 comprises a treatment head 2 with a unit to emit HIFU pulses, here in the form of a transducer 3. The transducer 3 is adapted to deliver HIFU pulses onto a target T in an object O. In the present embodiment, the treatment head 2 further comprises an imaging device 4. The treatment head further comprises a balloon 5 for receiving a fluid for acoustic coupling. Here, the device further comprises a movement device 6 that is connected to the treatment head 2 by an arm 7 and that is adapted to move the treatment head 2 along a longitudinal axis 8a, 8b. In particular, the movement device can be controlled to perform dynamic movements such as oscillations or movement with varying speeds. In the present embodiment, the movement device 6 also comprises a mechanical range limiter 9a, 9b. The range limiters, which could also be embodied as electronic or software-based limiters, limit the accessible range of the treatment head in order to prevent damage to the patient or the device. The device also comprises a controller unit 10 that is operatively connected to the transducer 2 and the movement device 6, in the present example by means of a cable 11.

FIG. 2 schematically shows how the method according to the invention works. A treatment head 2 comprising a balloon 2 is used to treat a target in an object O. Here, the method is carried out before the treatment of a patient. However, it is also possible to conduct the same method during a treatment. First, a longitudinal axis 8a is defined. Here, the longitudinal axis corresponds to the main propagation direction of the HIFU pulses, however, another axis, for example orthogonally to the main ultrasound propagation axis 8b, could be chosen as well. Using a movement device (not shown) that is operatively connected to the treatment head via an arm 7, the treatment head 2 is moved along the longitudinal axis 8a, 8b while operating an imaging device (not shown) integrated in the treatment head. The imaging device records several images 12a, 12b, 12c, 12d, 12e at different locations that correspond to different points along the longitudinal axis 8a, 8b. Here, the target within the object O is visible only on some of the collected images. On the images 12b and 12d, the target T is visible as representation 13c and 13a. On image 12c, the target T is only partially visible 13b. The collected images can later be accessed by the operator and used to locate the target. Alternatively, the same images could also be collected during treatment if the target T becomes invisible in the ultrasound image.

FIG. 3 shows an example of a user interface 39 with a set of acquired images 31. The user intends to perform a sonication in the slice displayed in the live view 34. In this slice, the precise position, symbolized by arrows 33, of the vein is difficult to determine. Therefore, the user scrolls within the spatial cine-loop 1 with the slider 32 to a neighbouring position where the vein clearly can be seen. Finally, the user mentally follows the vein as when scrolling back to the slice where a sonication is to be performed.

FIG. 4 shows an illustration of a possible user interface 39. In this example, the treatment head has been moved laterally after the acquisition. Thus, the images of the set of acquired images 31 have been shifted, which results in the yellow area 40, where no information is available for display. In this example, a button 36 enables to place a marker 35 on the images of the set of acquired images 1. As the two images are spatially consistent due to the shifting, the marker 35 is simply displayed in the live view 34 at the same position in the image. In this example, a button to hide the marker 38 is added to the interface. This allows for better visualization since markers, although helpful, may hinder visibility of the anatomical structures. In addition, a user may delete a marker with a third button 37.

FIG. 5 shows schematically a rotational movement of the treatment head 2. Initially, the treatment head oriented at a target T in a patient's body (not shown). The focal spot 16 of the HIFU beam 15 is positioned at a site that is to be treated. However, from that particular angle, the target may not be very well visible. Thus, the treatment head is rotated 17 around and axis 14 that intersects with the focal spot 16. Because the focal spot lies on the rotational axis, it does not move. The treatment head 2 and the HIFU beam 15 rotate around the axis such that the orientation remains the same and toward the axis 14.

FIG. 6 schematically shows an embodiment of the working principle of reference positions. A user may have saved several reference positions along a vein (not shown). The user interface is adapted to display several lines 19, each representing a reference position along the direction 20 of the vein. Additionally, the user interface also displays the position of the treatment head. However, at that position, the vein may not be visible. Thus, the user clicks on a position 18 where he wishes to view the vein. The device automatically calculates which reference position 19 is closest to the position 18 the user clicked on. The treatment head 2 is then moved to that position. The user can then examine the vein because of its increased visibility at the new position. He may then choose to move back by clicking in the vicinity of the treatment position 21. The device calculates the closest reference position, which in this case is the treatment position, and moves the treatment head there.

FIGS. 7a-7d show schematically a movement of the treatment head (not shown).

FIG. 7a shows the focus point 100 of an ultrasonic treatment head (not shown) in tissue 101. A target T is located in the tissue 101 and not visible in the ultrasonic image at the shown arrangement of the focal spot 100. The focus point 100 is displaced from the target T by a distance 102. However, the user cannot be aware of the displacement 102 due to the lack of visibility of the target T. The image taken by the treatment head in FIG. 7a is a cross-section in a first plane P1. In order to make the target T visible, the treatment head is moved from a first position along a longitudinal axis of the target T (i.e. along an axis perpendicular to the plane of the drawing shown here) to a second position such as to image a cross-section in a plane P2 (see FIG. 7b).

FIG. 7b shows the recorded image in the plane P2. Plane P2 is parallel to plane P1 of FIG. 7a, but displaced along the longitudinal axis of the target T. In plane P2, the target T is visible. Therefore, the user can see that the focus point 100 is laterally displaced by distance 102 from the target T.

As shown in FIG. 7c, the user thus moves the treatment head such as to position the focus point in 100 onto the target in plane P2 by laterally displacing the treatment head. Here, the user performs this movement manually and positions the treatment head such that the focus 100 falls onto the target T. Alternatively, however, it would also be possible to automatically move the treatment head or only measure the distance 102 for calculation of a corrected reference position (see FIG. 7d).

FIG. 7d shows the focus point 100 that has been moved back along the longitudinal axis of the target T to a corrected reference position. Here, the corrected reference position refers to a position at the same position along the longitudinal axis of the target T, but displaced in a direction parallel to the planes P1, P2 by a distance 102 that corresponds to the displacement between the target T and the focus 100 in FIG. 7a. Therefore, the focus point 100 in FIG. 7d is located on the target T which is not visible in plane P1.

The person skilled in the art will note that the planes P1 and P2 correspond to an identical treatment orientation, i.e. the treatment head is oriented identically with respect to the target in the FIGS. 7a-7d. Therefore, the reference position and the corrected reference position are defined with respect to the same treatment orientation (i.e. the planes P1 and P2 remain parallel).

1-33. (canceled)

34. A device for treatment of a patient by HIFU, the device comprising
a treatment head including a unit for emission of HIFU pulses,
an imaging device having a probe
a control unit for controlling the movement of the probe
wherein the control unit is adapted to carry out a movement of the probe with respect to a target (T) during operation of the imaging device.

35. The device according to claim 34, wherein the control unit is adapted to allow a user-controlled movement and/or to carry out a movement of the probe which approximately follows one of the following axes:
axis orthogonal to the current imaging plane
axis parallel to the main axis of the target
projection of the main target axis in a plane orthogonal to the main ultrasound propagation axis
projection of the main target axis in a plane parallel to the skin surface.

36. The device according to claim 35, wherein the control unit is adapted to store at least one reference position in a memory, and wherein the control unit is further adapted to trigger a movement of the probe to the reference position.

37. The device according to claim 34, wherein the control unit is adapted to carry out a movement of the probe away from an initial position at a first speed, followed by a return.

38. The device according to claim 34, wherein the device comprises a user interface to trigger a movement of the probe away from the current position.

39. The device according to claim 38, wherein the user interface comprises at least one actuator to trigger a movement of the probe away.

40. The device according to claim 38, wherein the user interface comprises two buttons to trigger a movement of the probe, one for each direction along a chosen axis.

41. The device according to claim 38, where the at least one button is selected from the group of a physical or virtual button.

42. The device according to claim 34, wherein the control unit is adapted to carry out an oscillatory movement of the probe roughly along one of the aforementioned axes.

43. The device according to claim 42, wherein the control unit is adapted to carry out an oscillatory movement selected from the group of
one or a predefined number of oscillations around an initial position
a finite number of damped oscillations around a current position
continuous oscillations around an initial position until a predetermined criterion is fulfilled.

44. The device according to claim 43, wherein the control unit is adapted to carry out an oscillatory movement with an amplitude greater than 1 mm.

45. The device according to claim 34, wherein the control unit is adapted to carry out a movement along an at least partially curved trajectory.

46. The device according to claim 45, comprising at least one of an input interface for definition of the trajectory by the user and a calculation unit for automatically computing the trajectory.

47. The device according to claim 34, wherein the control unit is adapted to carry out the movement by a control representing the coordinates of the probe.

48. The device according to claim 34, comprising a range limiter for limiting the movement of the probe.

49. The device according to claim 48, wherein the range limiter is defined by mechanical limits of a holding arrangement for holding the probe or definable through a user interface.

50. The device according to claim 34, wherein the device is adapted to associate the collected images to a coordinate along the trajectory and wherein the device comprises a display adapted to display the image corresponding to a given coordinate.

51. The device according to claim 50 wherein the display comprises a shared area for displaying the acquired images and for are for display of an image of the zone where a sonication has to be delivered, whereby
a. when the virtual position of the probe is set at the actual position of the probe, the live image of the zone where a sonication has to be delivered is displayed
b. when the virtual position of the probe is set it at another position, the corresponding image from the set of acquired images is displayed.

52. The device according to claim 34, wherein the navigation control is monostable so that, when it is released, the virtual position of the probe goes back to the actual position of the probe.

53. A method of preparing a treatment of a patient with HIFU, comprising the steps:
Performing a movement with a treatment head away from a target site
Acquiring at least one image away from the target site
Defining the position of a target in the vicinity of the target site based on the at least one acquired image
Performing a movement of the treatment head to the target site

54. A method of preparing a treatment of a patient with HIFU, comprising the acquisition of a collection of 2D prior to or during treatment, wherein the image acquisition comprises the following steps
Positioning a treatment head on the patient
Defining a longitudinal direction, wherein this direction,
Performing an automatic controlled movement along the longitudinal axis to acquire a set of images.
